(19) United States
(12) Patent Application Publication
(10) Pub. No.: US 2013/0298328 A1
(43) Pub. Date: Nov. 14, 2013

(54) POSITIONING APPARATUS FOR POSITIONING A PATIENT IN MEDICAL IMAGING SYSTEM

(71) Applicant: General Electric Company, Schenectady, NY (US)

(72) Inventor: Rajender Singh, Bangalore (IN)

(73) Assignee: General Electric Company, Schenectady, NY (US)

(21) Appl. No.: 13/890,588

(22) Filed: May 9, 2013

(30) Foreign Application Priority Data
May 11, 2012 (IN) 1886/CHE/2012

(51) Int. Cl. A61B 6/04

(52) U.S. Cl. CPC A61B 6/0407
USPC 5/601; 29/428

(57) ABSTRACT

A positioning apparatus for positioning a patient in a medical imaging system, the positioning apparatus comprising at least one base unit, wherein a base unit of the at least one base unit comprises at least one driving unit, a table assembly configured to hold the patient, and at least one supporting unit connecting the table assembly and the base unit. A supporting unit of the at least one supporting unit is positioned below the table assembly to a substantial middle portion of the table assembly, wherein the at least one driving unit is configured to drive the supporting unit in a horizontal direction with respect to the base unit to move a portion of the table assembly into a scanning unit of the medical imaging system for positioning the patient.

### TECHNICAL FIELD

Embodiments of the present invention relate to positioning a patient in a medical imaging system and, more specifically, to a positioning apparatus for positioning a patient in a medical imaging system.

### BACKGROUND OF THE INVENTION

Medical imaging systems are commonly used for performing diagnostic activities on a patient's body. The medical imaging systems may be for example but not limited to a Computed Tomography (CT) imaging system, a Positron Emission Tomography (PET) imaging system, a Magnetic Resonance Imaging (MRI) system, a PET/CT imaging system X-Ray imaging system, Single Photon Emission tomography (SPECT) imaging system and a PET/MRI imaging system. The diagnostic activities may be conducted to identify any tumor or lesions on the patient's body. These tumors or lesions can be identified accurately only when the patient is positioned accurately with respect to a medical imaging system. A positioning table connected to the medical imaging system is usually used for positioning the patient.

The patient is allowed to lie down on a cradle of the positioning table. The cradle is supported by a supporting structure and the supporting structure includes driving mechanisms to facilitate the cradle to move in a horizontal direction. The cradle is allowed to move into a scanning unit or a bore of the medical imaging system for capturing medical images of the patient's body. The medical images may include but are not limited to functional images and anatomical images. As the supporting structure does not enter the scanning unit, the cradle may extend to position the patient within the scanning unit. The cradle extends to act as a cantilever. The patient's center of gravity also changes when the cradle extends. Due this cantilever configuration and weight of the patient, the cradle may have uncontrolled deflection with respect to a scan plane. The medical imaging system may be able to detect the size of the tumor or lesion, but due to this deflection the medical imaging system may not accurately identify the position of the tumor or lesion in the patient's body. As the position of the tumor or lesion may not be accurately identified and marked, a schedule of radiation therapy used for treating the tumor or lesion cannot be effectively planned. The difficulty of planning arises because the radiation therapy procedure is conducted in a rigid flat table where the patient is allowed to lie down and undergo the treatment. So as the images of the patient's body are captured with a deflection in the cradle, conducting a radiation therapy on the patient using a rigid flat table may be a less effective treatment.

Thus, there is a need for a positioning apparatus that conveniently positions a patient with respect to a medical imaging system.

### BRIEF DESCRIPTION OF THE INVENTION

The above-mentioned shortcomings, disadvantages and problems are addressed herein which will be understood by reading and understanding the following specification.

In an embodiment a positioning apparatus for positioning a patient in a medical imaging system is disclosed. The positioning apparatus includes one or more base units. At least one base unit may comprise one or more driving units, a table assembly and one or more supporting units. The table assembly may be used for holding the patient. The one or more supporting units may connect the table assembly and the base unit. A supporting unit is capable of being driven by a driving unit in a horizontal direction with respect to the base unit to move a portion of the table assembly into a scanning unit of the medical imaging system for positioning the patient.

In an embodiment a medical imaging system is disclosed. The medical imaging system includes one or more scanning units, a positioning apparatus, one or more base units, a table assembly and one or more supporting units. The one or more scanning units capture a medical image of a patient positioned with respect to the medical imaging system. The positioning apparatus includes one or more base units, a table assembly and one or more supporting units. The table assembly is capable of holding the patient. The supporting units are capable of connecting the table assembly and the base unit. A supporting unit is driven by a driving unit present in a base unit. The supporting unit is driven in a horizontal direction with respect to the base unit to move a portion of the table assembly into a scanning unit of the at least one scanning unit for positioning the patient to capture the medical image.

In an embodiment method of assembling a positioning apparatus for positioning a patient in a medical imaging system is disclosed. The method comprises configuring a table assembly capable of holding a patient by connecting the table assembly to one or more base units using one or more supporting units. Then disposing one or more driving units connecting the supporting units on a base unit. A driving unit drives a supporting unit in a horizontal direction with respect to the base unit to move a portion of the table assembly into a scanning unit of the medical imaging system.

Various other features, objects, and advantages of the invention will be made apparent to those skilled in the art from the accompanying drawings and detailed description thereof.

### BRIEF DESCRIPTION OF THE DRAWINGS

FIG. 1 is a schematic illustration of a positioning apparatus for positioning a patient in a medical imaging system in accordance with an embodiment;

FIG. 2 is a schematic illustration of the positioning apparatus illustrated in FIG. 1;

FIG. 3 is a schematic illustration of a positioning apparatus for positioning a patient in a medical imaging system in accordance with another embodiment;

FIG. 4 is a schematic illustration of a medical imaging system in accordance with an embodiment; and

FIG. 5 illustrates a flow chart for assembling a positioning apparatus for positioning a patient in a medical imaging system in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

As discussed in detail below, embodiments of the present invention including a positioning apparatus for positioning a patient in a medical imaging system is disclosed. The positioning apparatus includes one or more base units. A base unit may have one or more driving units, a table assembly and one or more supporting units. The table assembly may be used to hold the patient. The one or more supporting units may connect the table assembly and the base unit. A supporting unit is driven by a driving unit in a horizontal direction and vertical direction with respect to the base unit to move a portion of the table assembly into a scanning unit of the medical imaging system for positioning the patient.

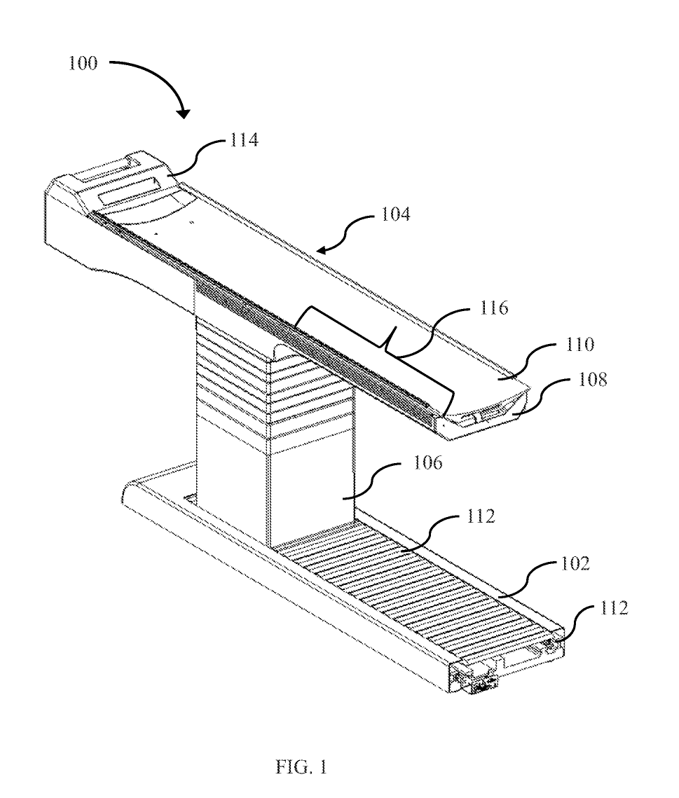

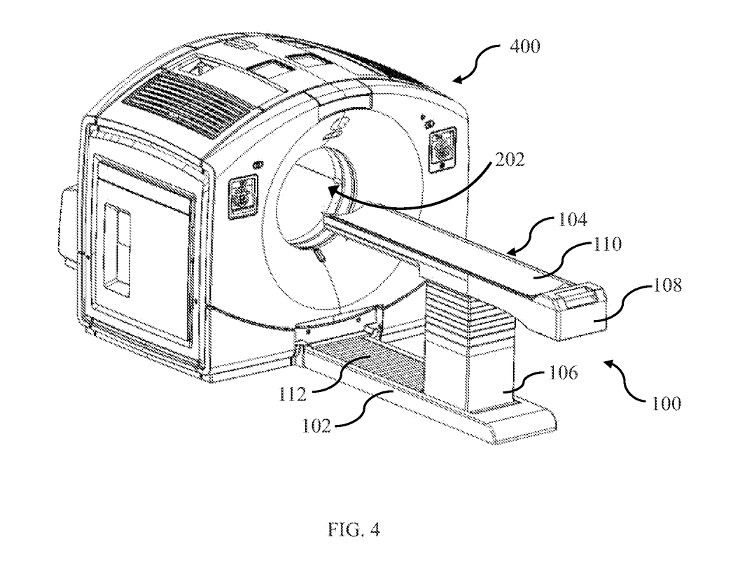

FIG. 1 is a schematic illustration of a positioning apparatus 100 for positioning a patient in a medical imaging system in accordance with an embodiment. The patient may be positioned with respect to the medical imaging system for capturing an image such as a medical image of a body part or whole body of the patient. Medical images may include but are not limited to functional images and anatomical images. The medical imaging system may include but not limited to a Computed Tomography (CT) imaging system, a Positron Emission Tomography (PET) imaging system, a Magnetic Resonance Imaging (MRI) system, PET/CT imaging system, a PET/MRI imaging system, X-ray imaging system and Single Photon Emission tomography (SPECT) imaging system. These medical imaging systems may be used to capture a CT image, a PET image, a MRI image, a co-registered PET/CT image, a X-ray image, a co-registered PET/MRI image and not limited to co-registered images of the images of the body part or the whole body of the patient. The images may be used to decide on a schedule associated with but not limited to, chemotherapy, radiation therapy and surgery for the patient. Radiation therapy is used to treat some diseases in the patient such as, cancer diseases. A high level of ionizing radiation such as, X-ray is exposed to an active tumor or lesion and this high dose radiation kills the active tissue. The exposed radiation also kills healthy tissue cells which come in its range of radiation. To reduce the harm caused to the healthy tissue cells it is important to accurately localize the lesion relative to the surrounding anatomy. The accuracy of these captured images depend on the capability of a positioning apparatus such as, the positioning apparatus 100 to precisely position the patient with respect to one or more scanning units of the medical imaging system. The positioning apparatus may be capable of moving the patient with respect to a scanning unit and position the patient for capturing images of various body parts of the patient. The scanning unit may have a bore like configuration that receives the patient there within for capturing the images. The configuration of the scanning unit is illustrated and explained in conjunction with respect to FIG. 4.

The positioning apparatus 100 includes one or more base units 102, a table assembly 104 and one or more supporting units 106. The table assembly 104 includes a cradle supporting structure 108 and a cradle 110 movably mounted on the cradle supporting structure 108. In an embodiment the cradle supporting structure 108 may include a guide way for enabling the cradle 110 to move with respect to the cradle supporting structure 108. The cradle supporting structure 108 may have a design and shape to enable the cradle supporting structure 108 to be positioned within a scanning unit of a medical imaging system. However the cradle supporting structure 108 and the cradle 110 may include any other mechanisms known in the art to facilitate the movement of the cradle 110. The cradle 110 may be but not limited to a curve table or a flat table or a flat table top integrated with the curve table. Further a supporting unit 106 connected to the table assembly 104 and the base unit 102 may also move with respect to the base unit 102. The supporting unit 106 may be positioned below the table assembly 104. The supporting unit 106 may be connected to a substantial middle portion of the table assembly 104. Thus the supporting unit 106 provides the support to reduce any cantilever effect on the table assembly 104 when the patient lies on the table assembly 104. However the supporting unit 106 may be positioned at any other position below the table assembly 104 to provide support. The base unit 102 may include a driving unit 112 that drives the supporting unit 106 in a horizontal direction. This horizontal movement in turn enables the table assembly 104 to move in the horizontal direction. The table assembly 104 also includes a handle unit 114 for moving the positioning apparatus 100 in case of any emergency.

Once the patient is positioned on the cradle 110, the cradle supporting structure 108 and the cradle 110 may be moved in a vertical direction using a lifting unit (not shown in FIG. 1) present in the supporting unit 106. The lifting unit is used to adjust the height of the table assembly 104 for aligning the table assembly with respect to the scanning unit 202. The height is adjusted such that the table assembly 104 can conveniently pass into the scanning unit, position the patient with respect to medical imaging system's iso-center and also allow the patient to conveniently lie down on or get down from the table assembly 104. The height of the table assembly 104 is adjusted to also facilitate in positioning imaging phantoms for performing calibration of the medical imaging system. The lifting unit may include but are not limited to a vertical actuating mechanism, a guide way mechanism, a lead screw mechanism or any other mechanisms known in the art. Thereafter the cradle 110 along with the cradle supporting structure 108 moves in order to align or position the patient with respect to the scanning unit of the medical imaging system.

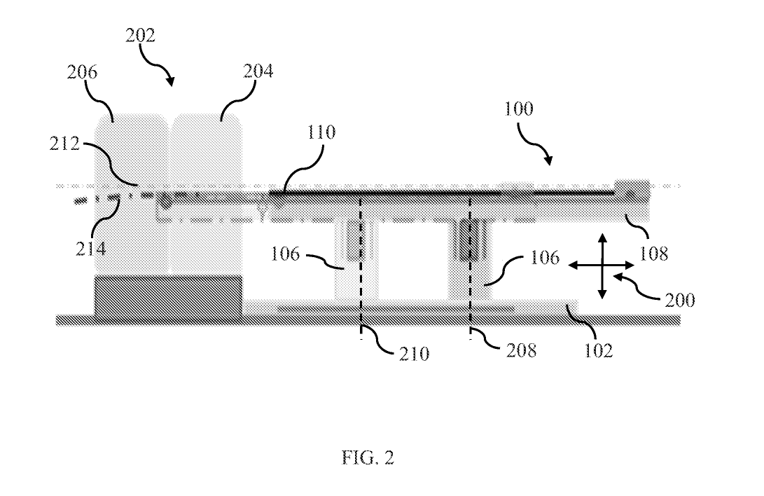

FIG. 2 is a schematic illustration of the positioning apparatus 100 for positioning the patient with respect to a scanning unit 202 in accordance with an embodiment. As illustrated in FIG. 2, the cradle 110 along with the cradle supporting structure 108 moves in a horizontal and vertical direction (depicted by arrows 200) in order to align or position the patient with respect to a scanning unit 202 of the medical imaging system. The scanning unit 202 may include a scanning section 204 and a scanning section 206. The scanning section 204 and the scanning section 206 may be positioned side by side. However the scanning sections may be positioned in any other manner or configuration.

The supporting unit 106 may move from a position 208 to a position 210 to move the cradle 110 from a scanning window of the scanning section 204 to a scanning window of the scanning section 206. A scanning window of a scanning section is an area exposed to the patient for capturing medical images of patient's whole body or body parts. As illustrated in FIG. 2, at the position 208 a portion of the cradle supporting structure 108 may enter a part of scanning section 204 to provide support to the cradle 110. More specifically the cradle supporting structure 108 may have a segment 116 and a portion of the segment 116 may enter a part of the scanning section 204 with a proximity to a scanning window of the scanning section 204 without interference of the cradle supporting structure 108 to the scanning window. The segment 116 may have a profile that is receivable within the scanning unit 202. The profile may be a thin configuration due to its reduced dimension and thickness. The reduced dimension enables the segment 116 to move into or accommodated within the scanning unit 202. The cradle 110 may then extend to expose the patient's body to the scanning window of the scanning section 204 for capturing the medical images. In an embodiment the cradle 110 extends with respect to the reference line 212 with reduced cantilever effect. The reduced cantilever effect is achieved due to the support provided by the cradle supporting structure 108.

Now to move to the scanning section 206 the cradle supporting structure 108 may move such that the segment 116 covers a substantial portion of the scanning unit 202. As illustrated in FIG. 2 the cradle supporting structure 108 may move parallel to the reference line 212 passing through the scanning section 204 to enter the scanning section 206. At this instance a segment of the segment 116 may enter the scanning section 206 with a proximity to a scanning window of the scanning section 206 without interference of the supporting structure 108 to the scanning window. Thereafter the cradle 110 may extend further without the support of the cradle supporting structure 108 to enter the scanning window of the scanning section 206. As a result the patient's body is exposed to the scanning window for capturing the medical images of the patient's body. An end portion of the cradle 110 that does not have the support may deflect from the reference line 212 as indicated by a deflect line 214. The end portion of the cradle 110 may act as a cantilever and deflect due to the own weight and the weight of the patient. However as the cradle supporting structure 108 provides support to the cradle 110 while covering the substantial portion of the scanning unit 202 the deflection of the cradle 110 is less in the respective active scanning window. This facilitates in precise positioning of the patient thereby leading to capturing of the medical images with more accuracy. Moreover due to the configuration of the segment 116 of the cradle supporting structure 108 the medical images may be captured at the scanning sections 204 and/or 206 with reduced cantilever deflection and the cantilever deflection of the cradle 110 may be uniform.

For example in a PET/CT imaging system, a scanning unit may include a PET scanning area and a CT scanning area. The PET and CT scanning areas may be positioned side by side in the PET/CT imaging system. However the PET and CT scanning areas may have any other configuration for capturing the images of the patient. In the imaging system a cradle and a cradle supporting structure may move within the scanning unit to position the patient for capturing images of a patient's body. The images taken may be a PET image and the CT image. The cradle supporting structure may be capable of moving such that a portion of the cradle supporting structure moves closer to a scanning window of a CT scanning area. The cradle may extend without the support of the cradle supporting structure to position the patient exposing to the CT scanning window for taking one or more CT images. The cradle may then move back to its normal position on the cradle supporting structure and subsequently the cradle supporting structure may move closer to a scanning window of a PET scanning area. Thereafter the cradle may extend without the support of the cradle supporting structure to position the patient to the PET scanning window for taking one or more PET images. As the cradle supporting structure moves closer to both the scanning areas there may be reduced cantilever deflection of the cradle and the deflection may be uniform while taking CT and PET images.

By avoiding cantilever deflection the patient may be positioned within respect to the scanning unit precisely thereby the PET and CT images captured may be accurate. So in the case of identifying tumor cells these co-registered PET and CT images may be capable of localizing the tumor cells with improved accuracy. The images may be also useful in performing improved Radiation Therapy planning in a convenient manner in order to treat the disease. As the radiation therapy is usually performed in a rigid flat-bed table, the images thus obtained by reduced cantilever deflection may be more useful for generating a plan for improved radiation therapy for the patients.

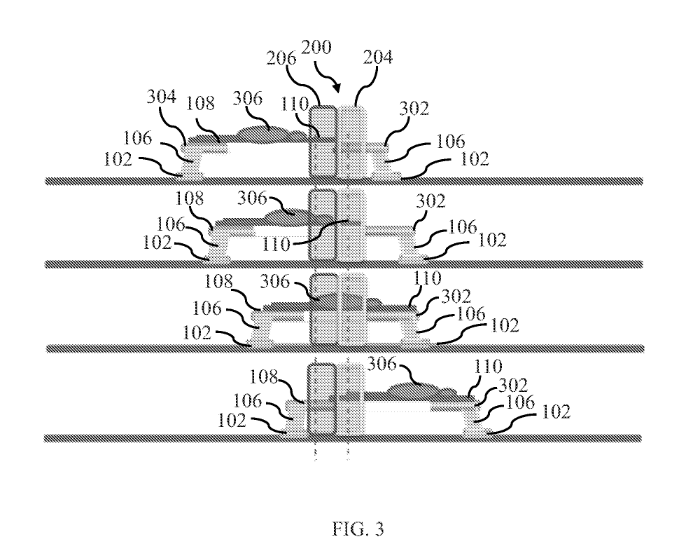

Referring now to the deflection in the cradle 110 that may occur while extending beyond the cradle supporting structure 108, a positioning apparatus such as positioning apparatus 100 may include a cradle receiving unit for receiving the cradle 110. FIG. 3 is a schematic illustration of a positioning apparatus 300 having a table assembly 302 including a cradle receiving unit 304. The cradle receiving unit 304 is connected to a supporting unit such as, a supporting unit 106 movably connected to the base unit 102. The base unit 102 may include a movement mechanism for moving the base unit 102 and the cradle receiving unit 304 in a horizontal direction. The cradle receiving unit 302 is connected to the supporting unit 106 movably mounted on a base unit such as, a base unit 102. In an embodiment a single base unit such as the base unit 102 may have two supporting units 106 connected to the cradle receiving unit 302 and the cradle supporting structure 108. Alternatively, separate base units such as a base unit 102 may be present to support the cradle receiving unit 302 and the cradle supporting structure 108. The supporting unit 106 moves in a horizontal and vertical direction with respect to the base unit to move a portion of the cradle receiving unit 302 into the scanning unit 202. In an embodiment the cradle receiving unit 302 may have a configuration similar to a configuration of the cradle supporting structure 108.

During operation when the cradle 110 moves through the scanning section 204 of the scanning unit 202 by extending beyond the cradle supporting structure 108, the cradle 110 is received by the cradle receiving unit 304. The cradle receiving unit 304 may move closer to a scanning window of the scanning section 204 from an opposite side to receive the cradle 110. The cradle receiving unit 304 provides support to the cradle 110 to reduce the cantilever deflection. The cradle supporting structure 108 may move closer to a scanning window of the scanning section 206. Subsequently the cradle 110 may extend to position a patient 306 to the scanning window scanning section 206. In this instance the cradle receiving unit 304 may move closer to the scanning window of the scanning section 206 from the opposite side to receive the cradle 110 to provide support.

Explaining by way of an example in a PET/CT imaging system, a scanning unit may include a PET scanning area and a CT scanning area. In the imaging system a cradle and a cradle supporting structure may move within the scanning unit to position the patient for capturing images of a patient's body. The images taken may be a PET image and the CT image. The cradle supporting structure may be capable of moving such that a portion of the cradle supporting structure moves closer to a scanning window of a CT scanning area. The cradle may extend without the support of the cradle supporting structure to position the patient exposing to the CT scanning window for taking one or more CT images. At this stage a cradle receiving unit may move closer to the scanning window of the CT scanning area from an opposite side to receive the cradle thereon. The cradle receiving unit may act as a support for an end portion of the cradle that extended thereby reducing the cantilever deflection of the cradle. The cradle may then move back to its normal position on the cradle supporting structure and subsequently the cradle supporting structure may move closer to a scanning window of a PET scanning area. Thereafter the cradle may extend without the support of the cradle supporting structure to position the patient to the PET scanning window for taking one or more PET images. In this instance the cradle receiving unit may move closer to the scanning window of the PET scanning area from the opposite side to receive the cradle thereon. As the cradle supporting structure and the cradle receiving unit move closer to both the scanning areas there may be continuous support to the cradle when a whole body scan of the patient is performed. Further there may be less occurrence of deflection of the cradle resulting in generation of better quality images.

Turning now to FIG. 4, FIG. 4 is a schematic illustration of a medical imaging system 400 having the positioning apparatus 100. As illustrated in FIG. 4 the positioning apparatus 100 is positioned at one side and connected to the medical imaging system 400. The positioning apparatus 100 includes the base unit 102, the table assembly 104 and the supporting unit 106. The table assembly 104 includes the cradle supporting structure 108 and the cradle 110 movably mounted on the cradle supporting structure 108. The supporting unit 106 connected to the table assembly 104 and the base unit 102, may also move with respect to the base unit 102. The base unit 102 may include a driving unit 112 that drives the supporting unit 106 in a horizontal direction with respect to the base unit 102. This horizontal movement in turn enables the table assembly 104 to move in the horizontal direction. Further the supporting unit 106 may include a lifting unit (not shown in the FIG. 4) for lifting and lowering the table assembly 106 in a vertical direction. When the table assembly 104 is parked or not operational the table assembly 104 is positioned at a distance from the medical imaging system 400.Once the patient is positioned on the cradle 110, the cradle 110 along with the cradle supporting structure 108 moves in a horizontal direction into the scanning unit 202 of the medical imaging system 400. Further a portion of the cradle 110 may move into the scanning unit 202 when driven by a cradle driving unit present in the cradle supporting structure 108. The scanning unit 202 may have a bore like structure as illustrated in FIG. 4. In an embodiment the scanning unit 202 may have multiple scanning areas. The scanning areas may be positioned side by side or in any other configurations known in the art. For example, in a PET/CT imaging system a PET scanning window and a CT scanning window may be positioned side by side. In another embodiment the scanning unit 202 may have a single scanning area. For example, a PET/CT imaging system may include a PET scanning window and a CT scanning window in the same location. In this case these scanning windows may overlap.

In an embodiment the scanning unit 202 may have a diameter of about 700 mm. However a scanning unit may have any other size suitable for its operation. The cradle supporting structure 108 may move to cover or trace a substantial portion of the scanning unit 202. Subsequently, the cradle 100 may extend to position the patient. In this scenario, the cradle receiving unit 302 (not shown in FIG. 4) may receive the cradle 100 that extends and provide support. The cradle receiving unit 302 may be placed on another side of the medical imaging system 400 opposite to the cradle supporting structure 108. The cradle receiving unit 302 may also move into the scanning unit 202 for receiving the cradle 100. The medical imaging system 400 and the positioning apparatus 100 may have provisions for programming the process of moving the cradle supporting structure 108 and the cradle receiving unit 302 so as to provide support to the cradle 110 carrying the patient and positioning the patient with respect to the medical imaging system 400.

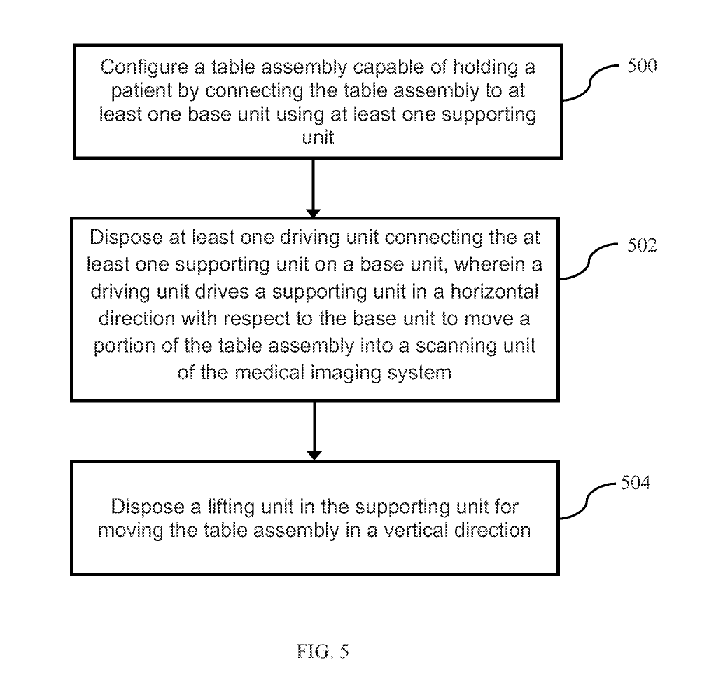

Now referring to FIG. 5, FIG. 5 illustrates a method of assembling a positioning apparatus such as the positioning apparatus 100 for positioning a patient in a medical imaging system in accordance with an embodiment of the present invention. The positioning apparatus is assembled and then placed proximal or connected to the medical imaging system. The positioning apparatus includes a table assembly, at least one supporting unit and one or more base units. For example the positioning apparatus 100 have the table assembly 104, the supporting unit 106 and the base unit 102 as explained in detail in conjunction with FIGS. 1, 2, 3 and 4. At step 500 the table assembly is configured to connect to the base units using the supporting units. The supporting units may be connected to the table assembly using any mechanism known in the art. In an embodiment the supporting unit is positioned below the table assembly in order to facilitate reduced cantilever deflection of a cradle present in the table assembly.

The supporting units and the base unit are connected to each other by disposing the driving units connecting the base unit and the supporting units at step 502. A driving unit drives or moves a supporting unit with respect to the base unit in a horizontal direction. As a result the table assembly is also moved in the horizontal direction. In order to move the table assembly in a vertical direction a lifting unit is disposed in a supporting unit at step 504. The lifting unit facilitates in elevating and lowering the table assembly. The table assembly is configured by mounting the cradle on a cradle supporting structure. In an embodiment the cradle supporting structure may include a guide way and a driving mechanism for facilitating the cradle to move with respect to the cradle supporting structure. However the cradle supporting structure and the cradle may include any other mechanisms known in the art to facilitate the movement of the cradle. The cradle supporting structure may have a segment with a profile that enables the segment to be received within the scanning unit. The profile may have a thin configuration due to reduced dimension and thickness. This segment of the cradle supporting structure covers a substantial portion of the scanning unit.

The positioning apparatus thus assembled may be connected or positioned proximal to the medical imaging system. The cradle, the cradle supporting structure and the supporting unit may be moved to position a portion of the cradle supporting structure within the scanning unit. The table assembly may hold a patient that may need to be positioned with respect to the scanning unit for capturing a medical image of the patient's body.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A positioning apparatus for positioning a patient in a medical imaging system, the positioning apparatus comprises:
at least one base unit, wherein a base unit of the at least one base unit comprises at least one driving unit;
a table assembly configured to hold the patient; and
at least one supporting unit connecting the table assembly and the base unit, wherein a supporting unit of the at least one supporting unit is positioned below the table assembly to a substantial middle portion of the table assembly, wherein the at least one driving unit is configured to drive the supporting unit in a horizontal direction with respect to the base unit to move a portion of the table assembly into a scanning unit of the medical imaging system for positioning the patient.

2. The positioning apparatus of claim 1, wherein the table assembly comprises:
a cradle supporting structure comprising a segment with a profile receivable within the scanning unit; and
a cradle movably mounted on the cradle supporting structure, wherein the segment of the cradle supporting structure and the cradle move into the scanning unit of the medical imaging system when driven by the at least one driving unit, wherein an end of the segment is configured to be positioned proximal to a scanning window of the scanning unit.

3. The positioning apparatus of claim 2, wherein the table assembly further comprises a cradle receiving unit positioned at a side of the scanning unit, wherein the cradle receiving unit receives thereon the cradle moving along the cradle supporting structure through the scanning unit, wherein the cradle receiving unit is supported by a supporting unit of the at least one supporting unit connected to a base unit of the at least one base unit, the cradle receiving unit is configured to be driven by a driving unit of the at least one driving unit for positioning a portion of the cradle receiving unit within the scanning unit of the medical imaging system.

4. The positioning apparatus of claim 1, wherein a supporting unit of the at least one supporting unit comprises a lifting unit configured to move the table assembly in a vertical direction with respect to the base unit.

5. A medical imaging system comprising:
at least one scanning unit configured to capture an image of a patient; and
a positioning apparatus comprises:
at least one base unit, wherein a base unit of the at least one base unit comprises at least one driving unit;
a table assembly configured to hold the patient; and
at least one supporting unit connecting the table assembly and the base unit, wherein a supporting unit of the at least one supporting unit is positioned below the table assembly to a substantial middle portion of the table assembly, wherein the at least one driving unit is configured to drive the supporting unit in a horizontal direction with respect to the base unit to move a portion of the table assembly into a scanning unit of the at least one scanning unit for positioning the patient to capture the image.

6. The medical imaging system of claim 5, wherein the table assembly comprises:
a cradle supporting structure comprising a segment with a profile receivable within the scanning unit; and
a cradle movably mounted on the cradle supporting structure, wherein the segment of the cradle supporting structure and the cradle move into the scanning unit of the medical imaging system when driven by the at least one driving unit, wherein an end of the segment is configured to be positioned proximal to a scanning window of the scanning unit.

7. The medical imaging system of claim 6, wherein the table assembly further comprises a cradle receiving unit positioned at a side of the scanning unit of the at least one scanning unit, wherein the cradle receiving unit receives thereon the cradle moving along the cradle supporting structure through the scanning unit, wherein the cradle receiving unit is supported by a supporting unit of the at least one supporting unit connected to a base unit of the at least one base unit, wherein the supporting unit is configured to be driven by a driving unit of the at least one driving unit for positioning a portion of the cradle receiving unit within a scanning unit of the at least one scanning unit.

8. The medical imaging system of claim 5, wherein a supporting unit of the at least one supporting unit comprises a lifting unit configured to move the table assembly in a vertical direction with respect to the base unit.

9. A method of assembling a positioning apparatus for positioning a patient in a medical imaging system, the method comprising:
configuring a table assembly configured to hold the patient by connecting the table assembly to at least one base unit using at least one supporting unit, wherein the at least one supporting unit is positioned below the table assembly to a substantially middle portion of the table assembly;
disposing at least one driving unit connecting the at least one supporting unit on a base unit of the at least one base unit, wherein a driving unit of the at least one driving unit drives a supporting unit of the at least one supporting unit in a horizontal direction with respect to the base unit to move a portion of the table assembly into a scanning unit of the medical imaging system; and
disposing a lifting unit in the supporting unit for moving the table assembly in a vertical direction.

10. The method of claim 9, wherein configuring the table assembly comprises movably mounting a cradle on a cradle supporting structure, wherein the cradle supporting structure comprises a segment with a profile receivable within the scanning unit, wherein the segment of the cradle supporting structure and the cradle are configured to move into the scanning unit when driven by the driving unit.
